(19) United States
(12) Patent Application Publication
(10) Pub. No.: US 2006/0263237 A1
(43) Pub. Date: Nov. 23, 2006

(54) Apparatus for steam sterilizing baby bottles

(75) Inventor: James W. Holley JR., Colorado Springs, CO (US)

Correspondence Address: BEVER HOFFMAN & HARMS, LLP;TRI-VALLEY OFFICE, 1432 CONCANNON BLVD., BLDG. G, LIVERMORE, CA, 94550, US

(73) Assignee: Insta-mix, Inc., Subsidiary A (DBA UMIX, Inc.), Colorado Springs, CO (US)

(21) Appl. No.: 11/131,655

(22) Filed: May 17, 2005

(51) Int. Cl. A61L 2/08

(52) U.S. Cl. 422/26

(57) ABSTRACT

A bottle sterilizer includes a housing assembly that attaches onto the neck of a bottle such that the bottle protrudes from the housing, and the bottle interior forms a part of the sterilization chamber. The housing assembly includes matching, substantially semi-cylindrical housing portions that have a lower opening to admit steam, and an upper opening communicates with an interior of the bottle. Other components of the bottle (e.g., nipple, collar, and cap) are mounted on ribs that are provided inside of the housing assembly. A sterilization assembly includes the housing assembly and a microwave base that is mounted under housing assembly when the steam sterilization procedure is performed using a microwave oven. A portable sterilization/warming system includes the housing assembly and a portable bottle warmer.

### FIELD OF THE INVENTION

This invention relates to sterilizer assemblies for sterilizing food containers for human use and, more particularly, to a sterilizer assembly especially adapted to sterilize baby bottles and parts thereof.

### BACKGROUND OF THE INVENTION

Sterilizers for baby bottles, nipples and other small parts are well known. Typically, two varieties of sterilizers have been used. A first, such as those shown in Schnell U.S. Pat. No. 2,467,337 and Meyerson U.S. Pat. No. 1,951,099, uses metallic pans containing metallic racks and covered by metallic covers for holding baby bottles and other items. A wire or other metallic support is placed within the pan to hold bottles, nipples and the like over a quantity of water. The pan is then covered and heated on a kitchen range or stove to create steam to cleanse and sterilize the items therein.

A second variety of sterilizer, such as those shown in Hoeck U.S. Pat. No. 4,544,529, is self-contained and includes its own electrical heating element along with a water container, rack and cover. Steam is created by heating the unit with the self-contained heating element.

A newer variety of sterilizers, such as those disclosed in Maniero U.S. Pat. No. 5,213,776, uses materials that are safe for microwave use (i.e., metallic parts are omitted). With these sterilizers, the baby bottles are disassembled, and the baby bottle parts (bottle, nipple, collar, and cap) are mounted either through an opening into the housing, or onto a base of the housing. A quantity of water is then put into the housing, and the housing is manipulated to enclose the baby bottle parts. The housing is then placed in a microwave oven, which is used to convert the water in the housing into steam, thus sterilizing the bottle and parts stored therein.

A problem with conventional sterilizer assemblies is that, because they are large enough to enclose at least one baby bottle and all of its parts, they are too bulky to conveniently carry in a conventional diaper bag. Therefore, on long trips away from home, a parent is often required to re-use a baby bottle that has not been sterilized.

What is needed is a compact bottle sterilizer assembly that can be conveniently carried, for example, in a diaper bag. What is also needed is a bottle sterilizer assembly that can be adapted for use either in a portable bottle warmer or in a microwave oven. What is also needed is a portable system that can be utilized both to sterilize baby bottles, and then to warm milk stored in the sterilized baby bottles.

### SUMMARY OF THE INVENTION

The present invention is directed to a bottle sterilizer housing assembly that attaches onto a neck of the bottle body such that the bottle body protrudes from the housing assembly, and the bottle interior forms a part of the sterilization chamber. By utilizing the bottle in this manner to increase the effective size of the sterilization chamber, the present invention facilitates the production of a sterilizer housing assembly that is about the size of a baby bottle, thus making the sterilizer housing assembly conveniently portable, for example, in a diaper bag.

In accordance with an embodiment of the present invention, the housing assembly includes matching, substantially semi-cylindrical housing portions that have a lower opening to admit steam, and an upper opening that mounts over the neck of a bottle such that the base of the bottle protrudes from the housing assembly, and the interior of the bottle communicates with an interior of the housing assembly. Other components of the bottle (e.g., nipple, collar, and cap) are mounted on ribs that are provided inside of the housing assembly. Steam is introduced into the housing assembly interior, for example, through the lower opening, to sterilize the nipple, collar, and cap, and passes from the housing assembly interior into the bottle, thus sterilizing both the bottle and bottle components. In one embodiment, side walls of the housing portions define openings to facilitate steam flow.

In accordance with an embodiment, a sterilization assembly includes the housing assembly (described above) and a microwave base that is mounted under housing assembly when the steam sterilization procedure is performed using a microwave oven. The microwave base includes a disk-shaped lower wall, a cylindrical outer wall defining an upper opening, and a cylindrical inner wall for holding a predetermined quantity of water. When the housing assembly is mounted over the microwave base and placed inside a microwave oven, microwave energy converts the water into steam that enters the housing assembly and bottle body in the manner described above. In one embodiment, the cylindrical outer wall is elongated to cover openings formed in lower portions of the substantially semi-cylindrical housing portions.

In accordance with an embodiment, a portable sterilization/warming system including the housing assembly (described above) and a portable bottle warmer. The portable bottle warmer includes a rigid plastic housing having an opening that is sized to receive either the housing assembly during steam sterilization, or the baby bottle to warm a beverage stored therein. A heat generating system of the portable warmer includes a valve/switch, a heat generator, and a removable fuel source. An ON/OFF switch provided on the plastic housing controls the valve/switch to pass fuel from the fuel source to the heat generator. Heat generated by the heat generator is either used to both heat water inserted into portable bottle warmer to generate steam during the sterilization process, or to warm formula or milk stored in the baby bottle.

### BRIEF DESCRIPTION OF THE DRAWINGS

These and other features, aspects and advantages of the present invention will become better understood with regard to the following description, appended claims, and accompanying drawings, where:

FIGS. 1(A) and 1(B) are exploded and assembled perspective views, respectively, showing a housing assembly for steam sterilizing baby bottles according to a simplified embodiment of the present invention;

FIG. 2 is a flow diagram depicting a simplified method for steam sterilizing a baby bottle according to another embodiment of the present invention;

FIGS. 3(A) and 3(B) are exploded and assembled front views, respectively showing a baby bottle assembly;

FIG. 4 is a perspective view showing the front (open) side of a first housing portion formed in accordance with a specific embodiment of the present invention;

FIG. 5 is a perspective view showing the back (closed) side of the first housing portion of FIG. 4;

FIG. 6 is a perspective view showing the front (open) side of a second housing portion formed in accordance with a specific embodiment of the present invention;

FIGS. 7(A), 7(B), 7(C), 7(D), 7(E) and 7(F) are top plan views showing the process of mounting the various components of the baby bottle assembly of FIG. 3(A) into the housing assembly of FIGS. 4-6 in accordance with another embodiment of the present invention;

FIGS. 8(A) and 8(B) are cross-sectional front views showing a sterilization assembly includes the housing assembly of FIGS. 4-6 and a microwave base in accordance with another embodiment of the present invention;

FIGS. 9(A) and 9(B) are cross-sectional front views depicting a portable sterilization/warming system including a portable bottle warmer 901 according to another embodiment of the present invention;

FIG. 10 is a simplified diagram showing a heat generating system of the portable bottle warmer of FIG. 9(A);

FIG. 11 is a perspective view showing the portable bottle warmer of FIG. 9(A) while warming the contents of a baby bottle;

FIG. 12 is a perspective view showing the front (open) side of a first housing portion formed in accordance with another specific embodiment of the present invention;

FIG. 13 is a perspective view showing the front (open) side of a second housing portion associated with the first housing portion of FIG. 12;

FIG. 14 is a perspective view showing a microwave base for use with the first and second housing portions of FIGS. 12 and 13;

FIG. 15 is a perspective view showing a dust cover for use with the first and second housing portions of FIGS. 12 and 13;

FIG. 16 is a perspective view showing a housing assembly including the parts shown in FIGS. 12-15; and

FIG. 17 is a top plan views showing a mounting arrangement of the various components of the baby bottle assembly of FIG. 3(A) into the housing portion of FIG. 12 in accordance with another aspect of the present invention.

### DETAILED DESCRIPTION OF THE DRAWINGS

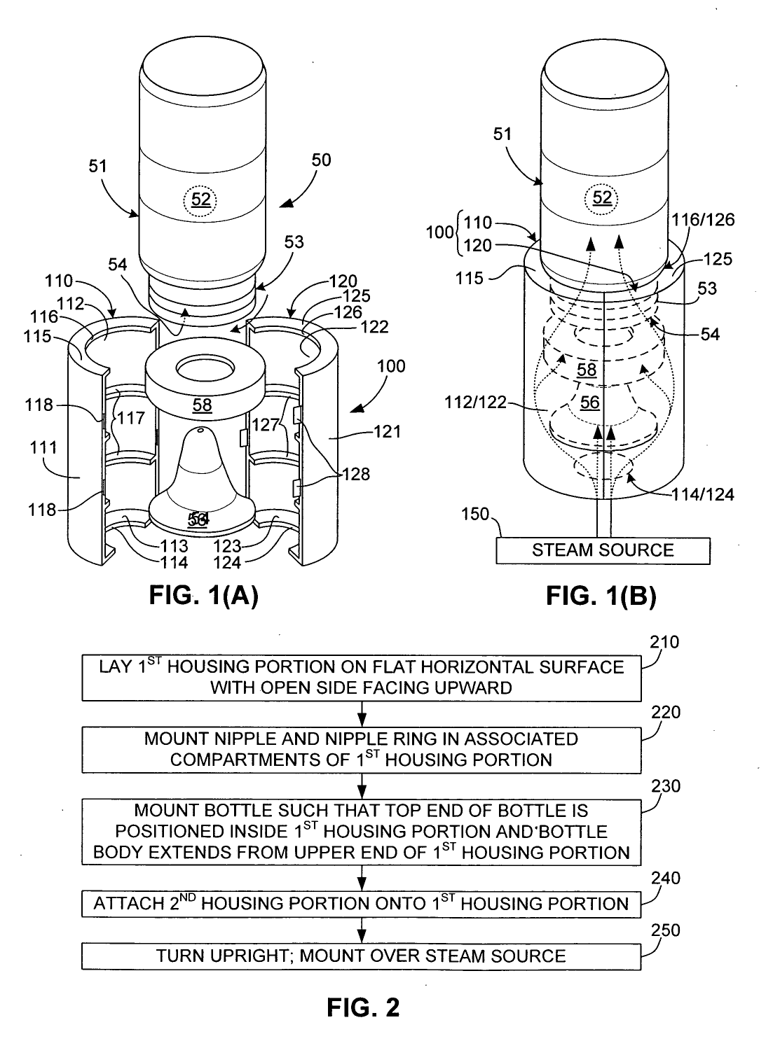

FIGS. 1(A) and 1(B) are exploded and assembled perspective views showing a simplified housing assembly 100 for steam sterilizing a baby bottle (bottle assembly) 50 according to an embodiment of the present invention.

Baby bottle 50 is a standard baby bottle that includes a bottle body 51 having an interior 52, a neck 53 located at an end of bottle body 51, and an opening 54 defined in neck 53 that facilitates the flow of liquid into and out of interior 52. Baby bottle 50 also including a “cover” made up of a nipple 56 and a collar 58 that operably attach to neck 53 in a well-known manner.

Housing assembly 100 includes a first housing portion 110 and a second housing portion 120 that are connected together in the manner described below. As indicated in FIG. 1(A), first housing portion 110 includes a semi-cylindrical housing wall 111 that defines a first inner chamber portion 112, a lower end wall 113 defining a lower opening portion 114, an upper end wall 115 defining an upper opening portion 116, and several ribs 117 extending from an inside surface of wall 111. Similarly, second housing portion 120 includes a semi-cylindrical housing wall 121 that defines a second inner chamber portion 122, a lower (second) end wall 123 defining a lower (second) opening portion 124, an upper (second) end wall 125 defining an upper (second) opening portion 126, and zero or more ribs 127 extending from an inside surface of wall 121.

When housing assembly is connected together, as indicated in FIG. 1(B), lower wall portions 113 and 123 collectively define a lower (first) opening 114/124, upper wall portions 115 and 125 collectively define an upper (second) opening 116/126, and semi-cylindrical housing walls 111 and 121 combine with the upper and lower walls to form a substantially cylindrical inner chamber 112/122. A suitable connecting mechanism, such as slots 118 defined in the edge of wall 111 that are sized to tightly receive tabs 128 extending from the edge of wall 121, are provided to detachably secure housing portion 110 to housing portion 120.

As indicated in FIG. 1(B), when housing assembly 100 is connected together in preparation to perform a sterilization procedure on baby bottle 50, upper wall portions 115 and 125 securely attach onto neck 53 such that bottle body 51 protrudes upside down from opening 116/126, and such that interior 52 of bottle body 51 communicates with inner chamber 112/122 of housing assembly 100. That is, opening 116/126 is sized such that upper wall 115/125 forms a tight seal around neck 53 in order to prevent unwanted leakage of steam during sterilization. In addition, ribs 117 and 127 combine to form shelves or compartments for separately supporting nipple 56 and collar 58 within inner chamber 112/122. In this manner, as indicated by the dashed-line arrows in FIG. 1(B), when steam is introduced from a steam source 150 through lower opening 114/124, the steam enters inner chamber 112/122 to sterilize nipple 56, collar 58, and neck 53, and also passes through opening 54 into bottle interior 52 to sterilize an inner surface of bottle body 51. An advantage of this arrangement is that the overall size of sterilizer housing assembly 100 is significantly smaller than conventional sterilizer apparatus because bottle body 51 forms a portion of the sterilization chamber. That is, because interior 52 of bottle body 51 communicates with inner chamber 112/122, the effective volume of the sterilization chamber is the sum of the volume of housing assembly 100 and the volume of bottle body 51. By eliminating the need for mounting bottle body 51 inside the sterilization chamber, housing assembly 100 can be made significantly smaller, thus allowing housing assembly 100 to be conveniently carried, for example, in a diaper bag.

FIG. 2 is a flow diagram depicting a simplified method for steam sterilizing baby bottle 50 according to another embodiment of the present invention. In general, the method involves mounting at least one of nipple 56 and collar 58 into inner chamber 112/122 of housing assembly 100 such that nipple 56 and collar 58 are separately supported inside inner chamber 112/122, and attaching neck 53 to housing assembly 100 such that bottle body 51 protrudes from housing assembly 100, and such that bottle interior 52 communicates with inner chamber 112/122 of housing assembly 100. In a presently preferred embodiment, this process involves laying first housing portion 110 on a horizontal surface such that the open side of first housing portion 110 is facing upward (block 210), mounting nipple 56 and collar 58 into associated compartments defined by ribs 117/127 (block 220), and mounting bottle body 51 such that a portion of neck 53 is resting on the inside edge of upper wall 115, with the free (open) end of neck 52 located over side wall 111 and the lower portion of bottle body 51 extending outside of first housing portion 110 and resting on the horizontal surface (block 230). With the various components stabilized in this state, second housing portion 120 is then aligned over first housing portion 110, and then attached securely to first housing portion 110, for example, by way of slots 118 and tabs 128 (block 240). Due to the secure connection between upper walls 115/125 and neck 52, housing assembly 100 and bottle body 110 can be manipulated as a single, integral unit (which nipple 56 and collar 58 secured inside), thereby facilitating turning the integral unit upright, and mounting the integral unit over a steam source 50 (block 250).

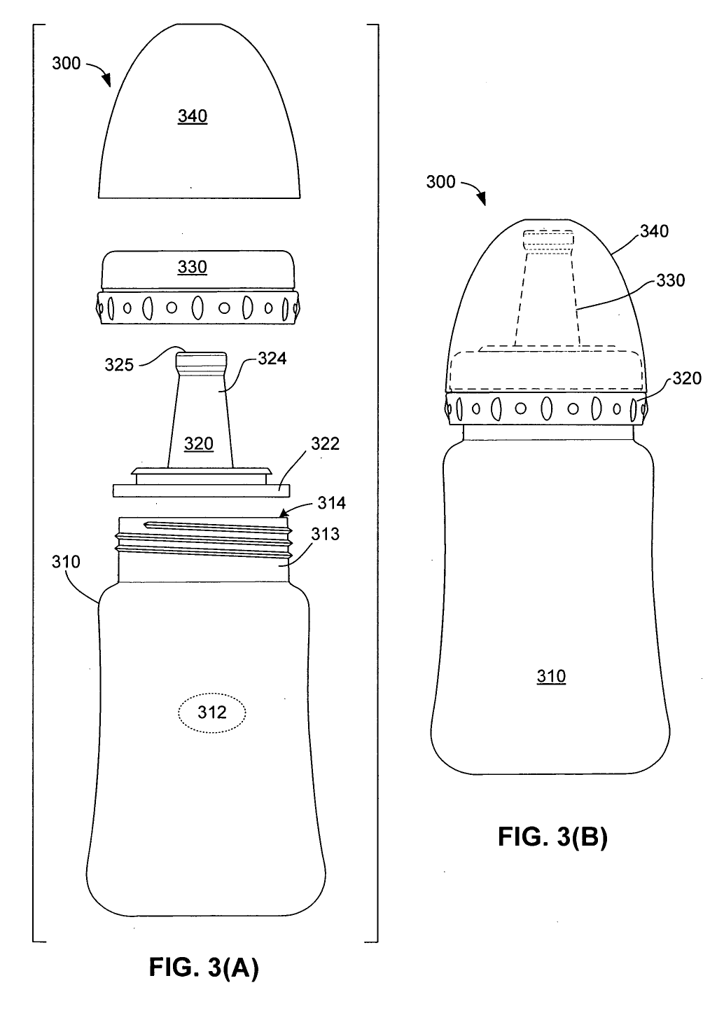

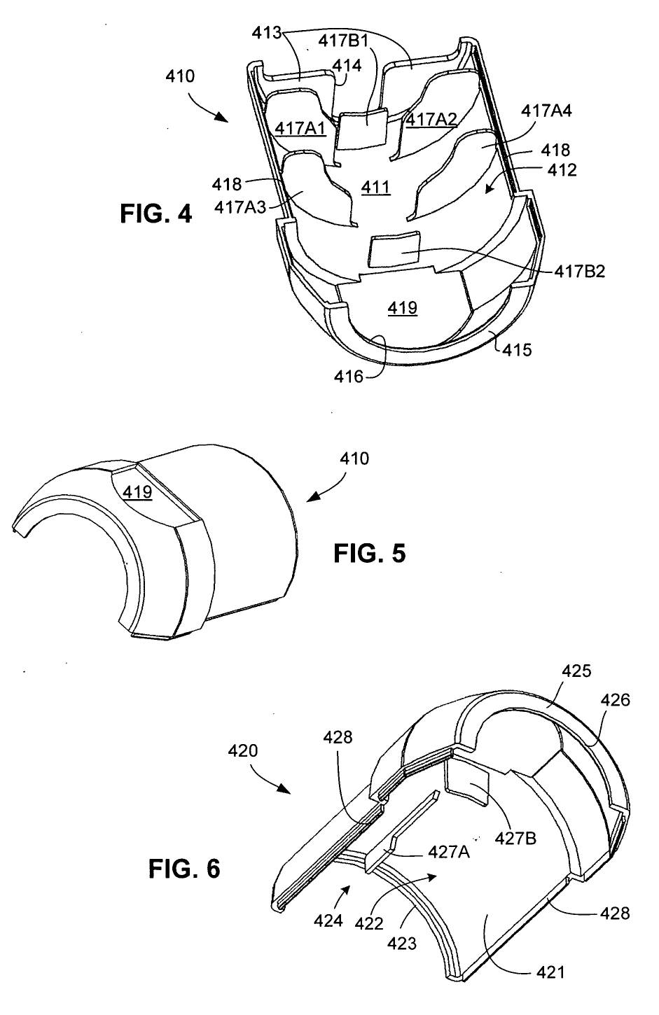

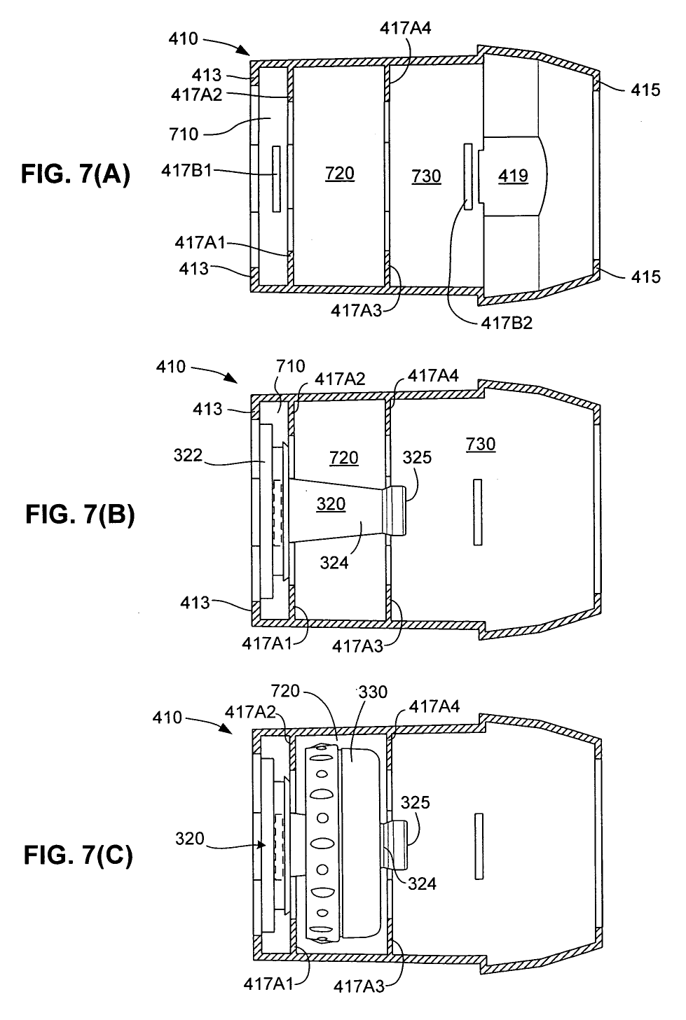

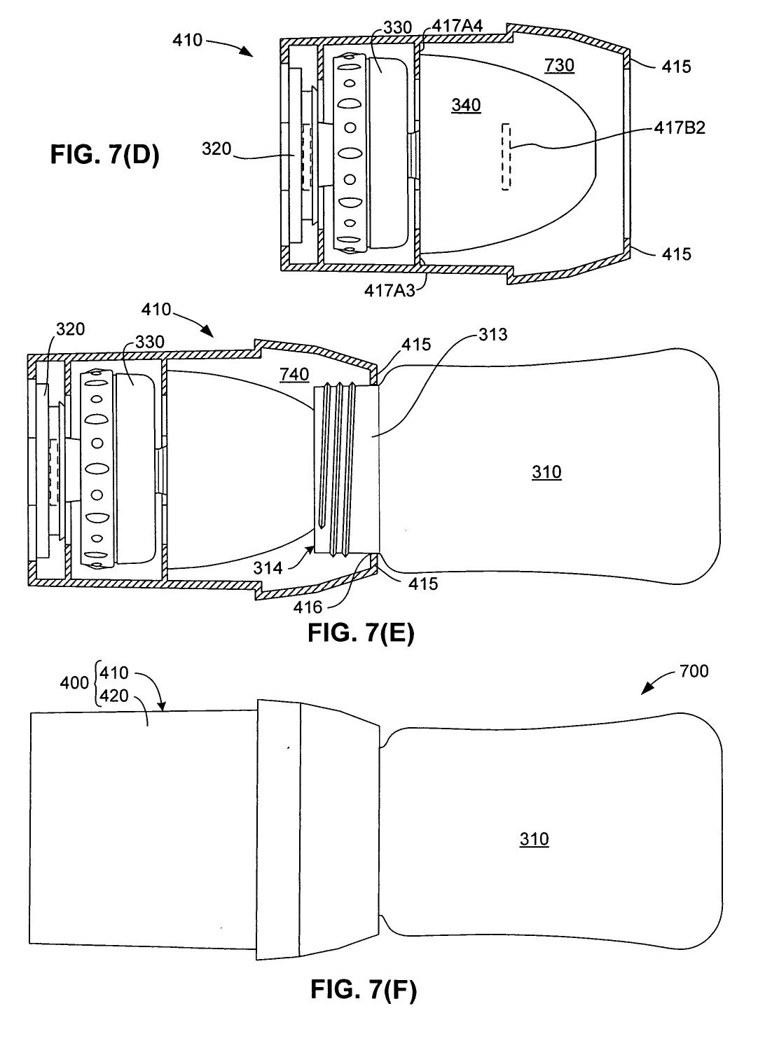

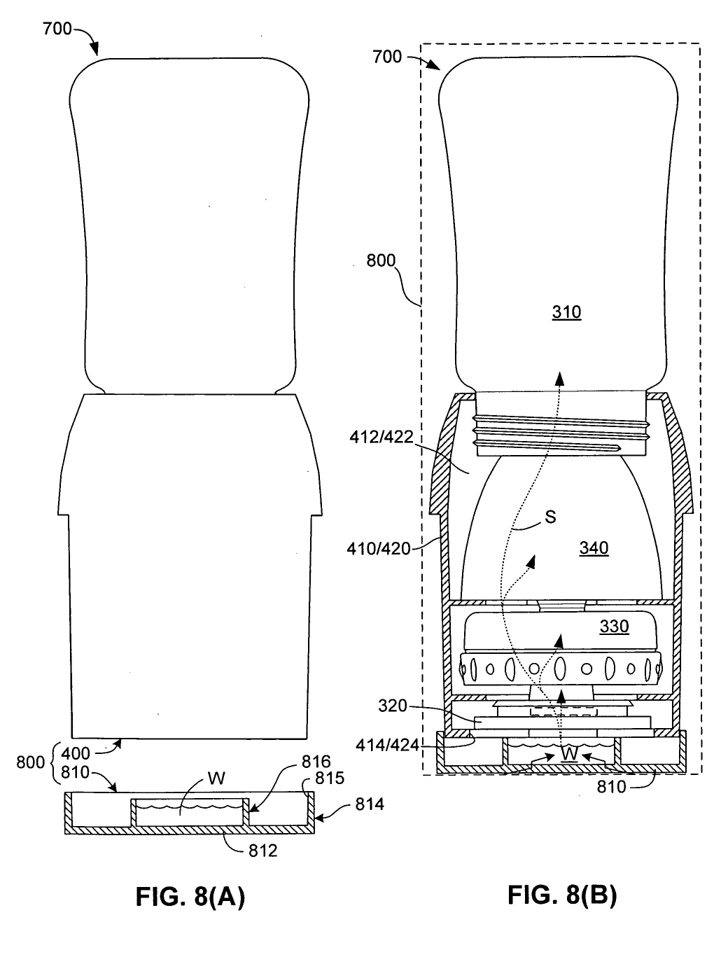

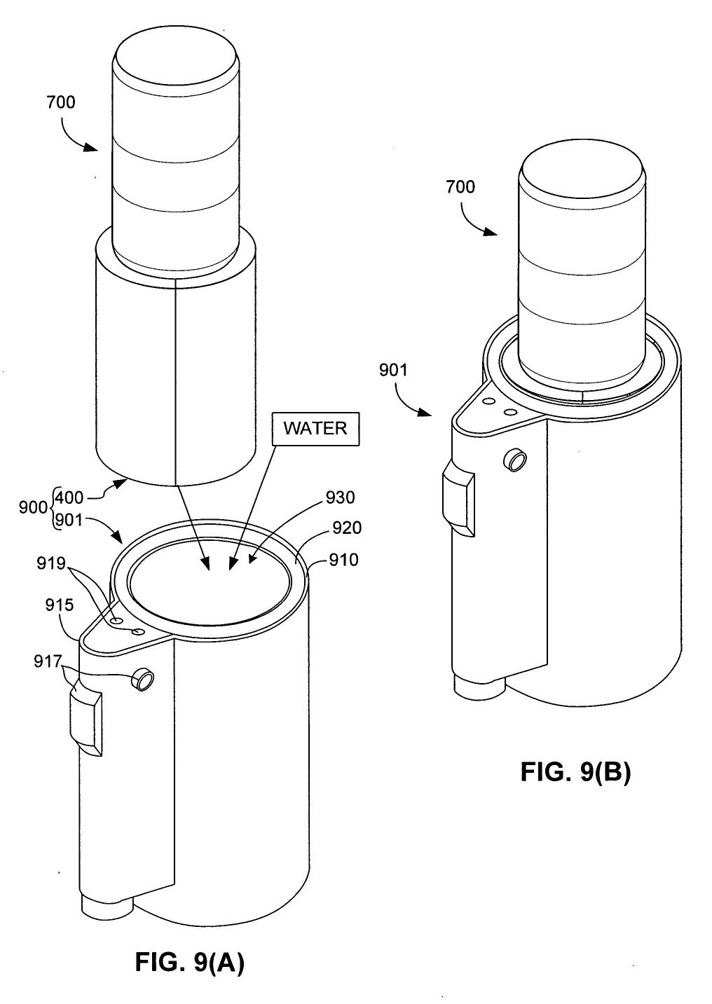

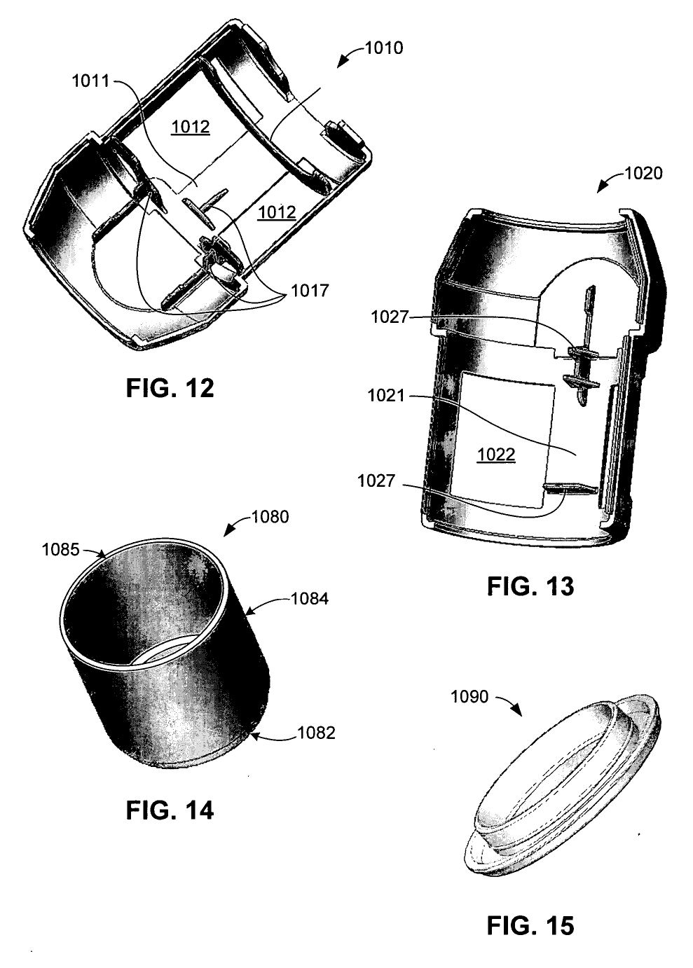

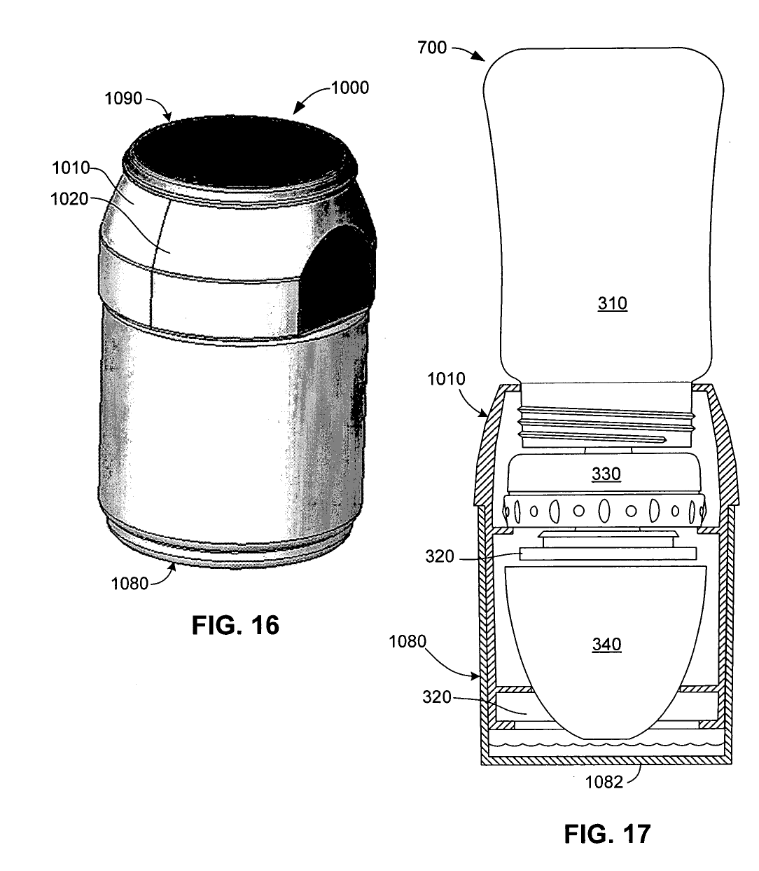

FIGS. 3 through 17 illustrate specific embodiments that depict additional features and aspects of the present invention. FIGS. 3(A) and 3(B) are exploded and assembled front views showing a baby bottle assembly 300 that is optimized for steam sterilization by a housing assembly formed using housing portions 410 and 420 depicted in FIGS. 4-6. FIGS. 7(A) through 7(F) are top plan views showing the process of mounting the various components of baby bottle assembly 300 into the housing assembly of FIGS. 4-6 in accordance with the method of FIG. 2. FIGS. 8(A) and 8(B) show an additional component of the housing assembly that is utilized with the integral bottle/housing assembly during steam sterilization using a microwave oven. FIGS. 9(A), 9(B), 10 and 11 show a portable system, including the housing assembly and a portable bottle warming apparatus, that make up another embodiment of the present invention. FIGS. 12-15 depict parts of a housing assembly 1000, which is shown in an assembled state in FIG. 16, and FIG. 17 shows the various components of baby bottle assembly 300 into housing assembly 1000.

Referring to FIGS. 3(A) and 3(B), baby bottle 300 includes a bottle body 310 having an interior 312, a neck 313 located at an end of bottle body 310, and an opening 314 defined in neck 313 that facilitates the flow of liquid into and out of interior 312. Baby bottle 300 also including a nipple 320, a ring-shaped collar 330, and a dome-shaped cap 340. Nipple 320 is formed from a relatively soft material (e.g., silicon), and includes a disk-like base 322, a tapered nozzle 324, and a flat end 325 that preferably includes a perforated membrane as described in co-owned and co-pending U.S. patent application Ser. No. 10/758,573, which is incorporated herein in its entirety. As indicated in FIG. 3(B), baby bottle 300 is assembled by mounting nipple 320 onto an upper edge of neck 313 over opening 314, and then screwing collar 330 onto threads provided on neck 313. Cap 340 is then snap coupled onto collar 330 to protect nipple 320 from contamination.

FIG. 4 is a perspective view showing the front (open) side of a first housing portion 410. First housing portion 410 includes a semi-cylindrical housing wall 411 that defines a first inner chamber portion 412, a lower end wall 413 defining a lower opening portion 414, an upper end wall 415 defining an upper opening portion 416, and several ribs 417A1-417A4 and tabs 417B1 and 417B2 extending from an inside surface of wall 411. First and second ribs 417A1 and 417A2 are co-planar and are separated by a groove that is provided to facilitate the passage of steam into other areas of the housing assembly. Similarly, third and fourth ribs 417A3 and 417A4 are also co-planar and are arranged parallel to first and second ribs 417A1 and 417A2, and also define a groove therebetween. Tabs 417B1 and 417B2 are provided to support associated bottle components during the mounting process, as described below. Slots 418 are formed in the edges of semi-cylindrical housing wall 411 to facilitate attachment to second housing portion 420. FIG. 5 shows the backside of first housing portion 410, and indicates a flat section 419 that serves to stabilize first housing portion 410 (i.e., resist rolling on a horizontal surface) during the mounting process described below. In one embodiment, an outer surface of flat section 419 is overmolded with rubber or high friction plastic.

FIG. 6 is a perspective view showing the front (open) side of a second housing portion 420 that operably connects with first housing portion 410 in the manner described below. Second housing portion 420 includes a semi-cylindrical housing wall 421 that defines an inner chamber portion 422, a lower edge 423 defining a lower opening portion 424, an upper end wall 425 defining an upper opening portion 426, and a longitudinal rib 427A and a tab 427B extending from an inside surface of wall 421. Longitudinal rib 427A and tab 427B serve to secure associated bottle components inside the housing assembly when second housing portion 420 is attached to first housing portion 410. Elongated protrusions 428 are formed along the edges of semi-cylindrical housing wall 421, and are tightly receives inside slots 418 to facilitate attachment of second housing portion 420 onto first housing portion 410. Like first housing portion 410, second housing portion 420 is fabricated using a rigid, heat tolerant and microwave safe material (e.g., plastic).

FIGS. 7(A) through 7(F) are top plan views showing the process of mounting the various components of baby bottle assembly 300 into first housing portion 410, and then the subsequent attachment of second housing portion 420 onto first housing portion 410. In these figures the uppermost surfaces of first housing portion 410 are shaded to facilitate understanding of the spatial relationships between the various parts and components.

FIG. 7(A) depicts first housing portion 410 upon being placed on a horizontal surface at the start of the mounting/assembly process. Note that flat section 419 contacts the horizontal surface (which lies parallel to the drawing sheet), and serves to resist rolling. Note also that first housing portion 410 defines several compartments for securing the various cover components of bottle 300 during the steam sterilization process. A first compartment 710 is defined between lower end wall 413 and parallel ribs 417A1 and 417A2, and first tab 417B1 is located inside compartment 710. A second compartment 720 is defined between parallel ribs 417A1/417A2 and parallel ribs 417A3/417A4. A third compartment 730 is defined between parallel ribs 417A3/417A4 and upper end wall 415.

FIG. 7(B) depicts first housing portion 410 after nipple 320 is mounted into first compartment 710. Base 322 of nipple 320 is securely held between lower wall 413 and parallel ribs 417A1/417A2, with nozzle 324 extending between the gap defined between parallel ribs 417A1/417A2, through second compartment 720, and into the gap formed between parallel ribs 417A3/417A4, with tip 325 located in third compartment 730. Note that nipple 320 is supported by tab 417B1 such that nozzle 324 is held in a substantially horizontal position to allow easy mounting of collar 330.

FIG. 7(C) depicts first housing portion 410 after collar 330 is mounted into second compartment 720, and is securely held between parallel ribs 417A1/417A2 and parallel ribs 417A3/417A4. Note that ring-shaped collar 330 defines a central opening that mounts over tip 325 and a portion of nozzle 324 of nipple 320.

FIG. 7(D) depicts first housing portion 410 after cap 340 is mounted into third compartment 730, and is held by parallel ribs 417A3/417A4 and tab 417B2. Note that cap 340 mounts over the end of nipple 320.

FIG. 7(E) depicts first housing portion 410 after bottle 340 is mounted such that neck 313 lays on upper end wall 415 and extends through upper opening portion 416 such that bottle opening 314 is located inside third compartment 340, and the end of cap 340 extends partially into bottle opening 314.

Finally, as shown in FIG. 7(F), second housing portion 420 is positioned over such that elongated protrusions 428 align with slots 418 of first housing portion 410 (shown in FIGS. 4 and 6, respectively), and second housing portion 420 is pressed downward such that elongated protrusions 428 become tightly received inside slots 418, thus attaching second housing portion 420 to first housing portion 410 and forming housing assembly 400. In addition, as described above, the action of attaching second housing portion 420 onto first housing portion 410 forms a bottle/housing assembly 700 that includes both assembly 400 and the various components of baby bottle 300. That is, bottle/housing assembly 700 includes nipple 320, collar 330, and cap 340 securely held inside housing assembly 400, and bottle body 310 securely held by housing assembly 400.

Once assembled as depicted in FIG. 7(F), bottle/housing assembly 700 can be manipulated as an integral unit to perform steam sterilization of baby bottle 300. According to another aspect of the present invention, housing assembly 400 facilitates steam sterilization both in a microwave oven, as illustrated below with reference to FIGS. 8(A) and 8(B), and also in conjunction with a portable bottle warmer, as illustrated below with reference to FIGS. 9(A), 9(B), 10 and 11.

Referring to FIG. 8(A), according to another embodiment of the present invention, a sterilization assembly 800 includes, in addition to housing assembly 400 (i.e., housing portions 410 and 420, described above), a microwave base 810 that is mounted under bottle/housing assembly 700 when the steam sterilization procedure is performed using a microwave oven. Microwave base 810 includes a disk-shaped lower wall 812, a cylindrical outer wall 814 defining an upper opening 815, and a cylindrical inner wall 816 for holding a predetermined quantity of water W (or other sterilizing liquid). As indicated in FIG. 8(B), when bottle/housing assembly 700 is mounted over microwave base 430 inside a microwave oven 800 (depicted by dashed box), microwave energy converts water W into steam S (depicted by dashed-line arrows), which enter inner chamber 412/422 of assembled housing portions 410/420 through lower opening 414/424, and passes into the various compartments by way of the grooves and other openings defined inside assembled housing portions 410/420, thereby sterilizing nipple 320, collar 330, and cap 340. In addition, steam S exits inner chamber 412/422 of assembled housing portions 410/420 and enters bottle body 310 in the manner described above. Note that venting grooves (not shown) are defined in outer cylindrical wall 814 to prevent excessive pressure from building up inside bottle/housing assembly 700.

FIGS. 9(A) and 9(B) depict a portable sterilization/warming system 900 including housing assembly 400 and a portable bottle warmer 901. Portable bottle warmer 901 includes a rigid plastic housing 910 formed around a cylindrical sleeve 920 that is formed from an elastic, thermally insulating material, such as neoprene, using known techniques. Housing 910 and cylindrical sleeve 120 define an opening 939 that is sized to receive either bottle/housing assembly 700 (as depicted in FIG. 9(B), or simply baby bottle 300 (as depicted in FIG. 11). Housing 910 includes a protrusion 915 for supporting a heat generating system (discussed below) such that the heat generating system is located outside of sleeve 920. Mounted on protrusion 915 are various control switches 917 and optional indicator lights 919.

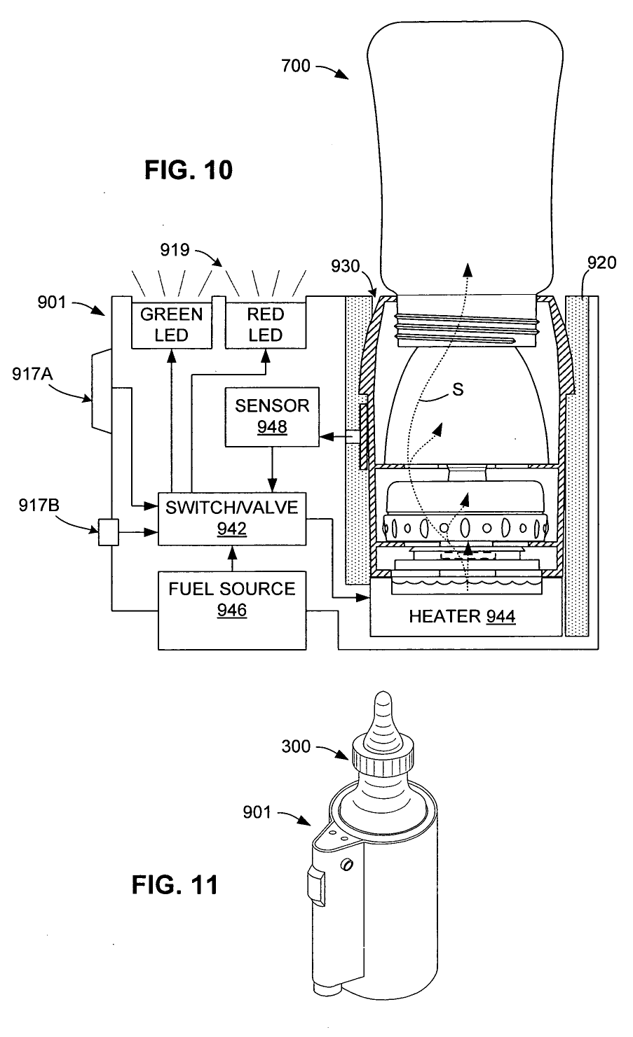

FIG. 10 is a simplified diagram showing a heat generating system of portable bottle warmer 901. The heat generating system includes a valve and/or switch 942, a heat generator 944 and a removable fuel source 946. Switch 942 is controlled by an ON/OFF switch 917A to pass fuel from fuel source 946 to heat generator 944. Heat generated by heat generator 944 is used to both heat water inserted into portable bottle warmer 901 to generate steam (as indicated in FIG. 10), and to warm formula or milk stored in baby bottle 300 (as indicated in FIG. 11).

In one embodiment, the heat generating system is a combustible gas burning system including a valve assembly (valve/switch 942) for passing a combustible gas (e.g., butane) from a removable container (fuel source) 942 to a gas-burning mechanism (heat generator) 944. A second switch 917A is provided to initiate gas flow from container 946 to gas-burning mechanism 944, and ON/OFF switch 117A facilitates ignition of the gas to produce a small gas flame in gas-burning mechanism 944. A suitable combustible gas burning system is disclosed in U.S. Pat. No. 4,699,123, entitled “Portable Heating Appliance”, which is incorporated herein by reference in its entirety. An optional heat sensor 948 is provided for turning off the flow of fuel from fuel source 946 to heat generator 944 when the steam sterilization process is completed, or when the baby bottle reaches a predetermined temperature. Different colored indicator lights (e.g., red and green light emitting diodes) 919 are provided to indicate the operating state.

FIGS. 12 to 15 are perspective views showing parts of a housing assembly 1000, which is shown in FIG. 16. FIG. 12 is a perspective view showing the front (open) side of a first housing portion 1010, which is similar to housing assembly 410 (described above) but includes a different arrangement of ribs 1017, and also defines side openings 1012 formed in semi-cylindrical housing wall 1011. FIG. 13 shows the front (open) side of a second housing portion 1020, which is similar to housing assembly 420 (described above) but includes a different arrangement of tabs/ribs 1027, and also defines side openings 1022 formed in semi-cylindrical housing wall 1021. According to an aspect of the present embodiment, side openings 1012 and 1022 facilitate steam flow when the assembly is mounted inside portable bottle warmer 901 (described above). FIG. 14 is a perspective view showing a cup-shaped microwave base 1080, which mounts over the lower sections of housing portions 1010 and 1020 (as depicted in FIG. 16) to facilitate steam sterilization using a microwave oven, and also to resist contamination during storage. Microwave base 1080 includes a disk-shaped lower wall 1082, and a cylindrical outer wall 1084 defining an upper opening 1085. FIG. 15 shows a dust cap 1090 that attaches over the upper end of the lower sections of housing portions 1010 and 1020 (as depicted in FIG. 16) to resist contamination during storage.

FIG. 17 is a top plan view showing a mounting arrangement of the various components of baby bottle assembly 300 into first housing portion 1010 according to another aspect of the present invention. The present inventor has determined that positioning cap 340 in the lower section of housing portion 1010 (e.g., adjacent to lower wall 1082 of microwave base 1080) may facilitate faster sterilization, and thus cap 340 is mounted below nipple 320 and collar 330 as shown in FIG. 17. As in previous embodiment, bottle body 310 extends from the upper end of housing portion 1010.

Although the present invention has been described with respect to certain specific embodiments, it will be clear to those skilled in the art that the inventive features of the present invention are applicable to other embodiments as well, all of which are intended to fall within the scope of the present invention.

1. An apparatus for steam sterilizing a bottle assembly, the bottle assembly including a bottle body defining a bottle interior, and a bottle neck located at an end of the bottle body, the bottle neck defining a bottle opening that communicates with the bottle interior, the bottle assembly also including a cover, the apparatus including a housing assembly comprising:
a housing wall defining an inner chamber;
support means located in the inner chamber for supporting the cap; and
attachment means for attaching to the bottle neck such that the bottle body protrudes from the housing assembly, and such that the bottle interior communicates with the inner chamber of the housing assembly, whereby steam introduced into the inner chamber enters the bottle interior through the bottle opening.

2. The apparatus of claim 1, wherein the housing assembly comprises:
a semi-cylindrical first housing portion including a first lower wall portion and a first upper wall portion; and
a semi-cylindrical second housing portion including a second lower wall portion and a second upper wall portion,
wherein the first and second lower wall portions collectively define a first opening for admitting steam into the inner chamber, and
wherein the first and second upper wall portions collectively form the attachment means, and collectively define a second opening for admitting steam from the inner chamber into the bottle interior.

3. The apparatus of claim 2, further comprising means for separately securing the first housing portion to the second housing portion.

4. The apparatus of claim 1, wherein the support means comprises a plurality of ribs formed on in inner surface of the housing wall.

5. The apparatus of claim 4,
wherein the cover of the bottle assembly includes a nipple and a collar for attaching the nipple over the bottle opening, and
wherein the plurality of ribs comprises a first rib for supporting the nipple, and a second rib for supporting the collar.

6. The apparatus of claim 5,
wherein the cover of the bottle assembly further includes a cap for attaching to the collar over the nipple, and
wherein the plurality of ribs comprises a third rib for supporting the cap.

7. The apparatus of claim 1,
wherein the housing assembly defines a first opening for admitting steam into the inner chamber, and
wherein the apparatus further comprises a microwave base for positioning an amount of water below the first opening.

8. The apparatus of claim 1, wherein the housing walls define openings.

9. A portable system for steam sterilizing a baby bottle assembly and for subsequently warming a beverage stored in the baby bottle assembly, the baby bottle assembly including a bottle body defining a bottle interior, and a bottle neck located at an end of the bottle body, the bottle neck defining a bottle opening that communicates with the bottle interior, the baby bottle assembly also including a nipple and a collar for securing the nipple to the bottle body over the bottle opening, the portable sysetm comprising:
a housing assembly including:
a housing wall defining an inner chamber and defining a first opening;
support means located in the inner chamber for supporting the nipple and the collar; and
attachment means for attaching to the bottle neck such that the bottle body protrudes from the housing assembly, and such that the bottle interior communicates with the inner chamber of the housing assembly; and
a portable bottle warmer including a housing defining a central opening that is sized to receive the housing assembly therein, the portable bottle warmer also including means for generating steam such that the steam enters the first opening of the housing assembly.

10. The portable system of claim 9, wherein the housing assembly comprises:
a semi-cylindrical first housing portion including a first lower wall portion and a first upper wall portion; and
a semi-cylindrical second housing portion including a second lower wall portion and a second upper wall portion,
wherein the first and second lower wall portions collectively define the first opening, and
wherein the first and second upper wall portions collectively form the attachment means, and collectively define a second opening for admitting steam from the inner chamber into the bottle interior.

11. The portable system of claim 10, further comprising means for securing the first housing portion to the second housing portion.

12. The portable system of claim 9, wherein the support means comprises a plurality of ribs formed on an inner surface of the housing wall.

13. The portable system of claim 12, wherein the plurality of ribs comprises a first rib for supporting the nipple, and a second rib for supporting the collar.

14. The portable system of claim 12,
wherein the cover of the bottle assembly further includes a cap for attaching to the collar over the nipple, and
wherein the plurality of ribs comprises a third rib for supporting the cap.

15. A method for steam sterilizing a baby bottle assembly, the baby bottle assembly including a bottle body defining a bottle interior, and a bottle neck located at an end of the bottle body, the bottle neck defining a bottle opening that communicates with the bottle interior, the baby bottle assembly also including a nipple and a collar for securing the nipple to the bottle body over the bottle opening, the method comprising:
mounting at least one of the nipple and the collar into an inner chamber defined by a housing assembly such that the nipple and collar are separately supported inside the inner chamber, and attaching the bottle neck to the housing assembly such that the bottle body protrudes from the housing assembly, and such that the bottle interior communicates with the inner chamber of the housing assembly; and
introducing steam into the inner chamber of the housing assembly such that the steam sterilizes the nipple and the collar, and such that the steam enters the bottle interior through the bottle opening and sterilizes and inside surface of the bottle.

16. The method according to claim 15,
wherein the housing assembly comprises a semi-cylindrical first housing portion including a first lower wall portion and a first upper wall portion, and a semi-cylindrical second housing portion including a second lower wall portion and a second upper wall portion, and
wherein mounting and attaching comprise connecting the first housing portion to the second housing portion such that the nipple and collar are located between the first and second lower wall portions and the first and second upper wall portions, and such that the bottle neck is held by the first and second upper wall portions.

17. The method according to claim 16, wherein mounting and attaching further comprises:
laying the first housing portion on a horizontal surface such that an open side of the first housing portion is facing upward;
mounting the nipple and collar into associated compartments defined in the first housing portion;
mounting the bottle body such that the bottle neck is positioned inside the first housing and the bottle body extends from the first housing; and
attaching the second housing portion onto the first housing portion.

18. The method according to claim 17,
wherein the first and second lower wall portions collectively define a first opening, and
wherein introducing steam comprises mounting the housing assembly and attached bottle body in an upright orientation over a steam source such that steam generated by the steam source enters the inner chamber through the first opening.

19. The method according to claim 18,
wherein the steam source comprises a microwave base for positioning an amount of water below the first opening, and
wherein introducing steam further comprises placing the microwave base, the housing assembly and the attached bottle body into a microwave oven.

20. The method according to claim 18,
wherein the steam source comprises a portable bottle warmer including a housing defining a central opening and means for generating steam located inside the central opening, and
wherein introducing steam further comprises pouring a predetermined amount of water into the central opening of the portable bottle warmer, and inserting the housing assembly into the central opening such that steam generated by the portable bottle warmer enters the first opening of the housing assembly.
